(19) United States
(12) Patent Application Publication
(10) Pub. No.: US 2026/0347828 A1
(43) Pub. Date: Nov. 5, 2026

(54) Pipeline for Separation and Detection of Ultrashort Single-Stranded Cell-Free DNA

(71) Applicants: The Regents of the University of California, Oakland, CA (US); National Cheng Kung University, Tainan (TW)

(72) Inventors: Jordan Cheng, Los Angeles, CA (US); David Wong, Pasadena, CA (US); Allen Huang, Tainan City (TW); Neeti Swarup, Los Angeles, CA (US); Irene Choi, Oakland, CA (US)

(21) Appl. No.: 19/486,618

(22) PCT Filed: May 23, 2024
(86) PCT No.: PCT/US2024/030795

§ 371 Date: Nov. 21, 2025

Related U.S. Application Data
(60) Provisional application No. 63/503,842, filed on May 23, 2023.

(51) Int. Cl. C12N 15/10; C12Q 1/6883; C12Q 1/6886

(52) U.S. Cl. CPC C12N 15/101; C12N 15/1013; C12N 15/1093; C12Q 1/6883; C12Q 1/6886; C12Q 2600/154; C12Q 2600/156

(57) ABSTRACT

A method of isolating ultrashort single-stranded cell-free DNA (uscfDNA) separately from other cell-free DNA, such as mononucleosomal cell-free DNA (mncfDNA) is described as well as methods of using the isolated mncfDNA and uscfDNA for detecting biomarkers and diagnosing diseases and disorders.

### CROSS REFERENCE TO RELATED APPLICATIONS

This application claims priority to U.S. Provisional Application No. 63/503,842, filed May 23, 2023, which is hereby incorporated by reference herein in its entirety.

### STATEMENT OF GOVERNMENT SUPPORT

This invention was made with government support under CA264398 and CA233370 awarded by the National Institutes of Health. The government has certain rights in the invention.

### BACKGROUND OF THE INVENTION

In liquid biopsy, cell-free DNA (cfDNA) analysis is typically focused on the mono-nucleosomal cfDNA (mncfDNA) biomarker of approximately 160 bp in length.

However, the current impression of the average fragment length of cfDNA is influenced by the inherent biases of nucleic acid extraction and library preparation. The recent adoption of single-stranded library preparation methods for cfDNA analysis suggests that in addition to mncfDNA, there are shorter cfDNA fragments (<100 bp) that can originate from either single-stranded or nicked dsDNA in plasma (Burnham et al., 2016; Snyder et al., 2016). Previous studies indicate that size-selecting for shorter fragments of cfDNA will enrich for mutant-containing cfDNA fragments in late-stage cancer patients (Mouliere and Rosenfeld, 2015). Next-generation sequencing approaches examining whole-genome differences in plasma cfDNA fragment lengths have revealed distinct fragment-profiles in cancer patients compared to those of healthy donors (Cristiano et al., 2019). Additionally, groups have attempted to utilize cfDNA strandedness as a diagnostic indicator (Huang et al., 2020; Zhu et al., 2020). With these considerations, ultrashort single-stranded cell-free DNA (uscfDNA) is an unexamined cfDNA entity with potential clinical relevance. In general, nucleic acid extraction kits are not designed to efficiently retain low-molecular cfDNA (<100 bp) regardless of strandedness (Diefenbach et al., 2018). Even when these extraction kits do retain low-molecular weight cfDNA, they are often contaminated with mncfDNA.

Thus, there remains a need in the art for an effective method for separation of different DNA species including uscfDNA and mncfDNA. This invention stratifies the unmet needs.

### SUMMARY OF THE INVENTION

The present invention provides a method of separately isolating mono-nucleosomal cfDNA (mncfDNA) and ultrashort single-stranded cell-free DNA (uscfDNA) molecules from a mixed nucleic acid sample, the method comprising the steps of: a) filtering the sample through a first silica column in the presence of reagents for DNA extraction and purification, wherein the mncfDNA is captured on the column and uscfDNA passes through the column into the flowthrough, and optionally eluting the mncfDNA off the column for downstream analysis; b) filtering at least a portion of the flowthrough of the filtration of step a) through a second silica column, without additional reagents; c) adding isopropanol to the flowthrough of the filtration of step b); d) applying the flowthrough of step c) to a third silica column in the presence of reagents optimized for purification and extraction of microRNA molecules, wherein the uscfDNA is bound to the column; e) eluting the uscfDNA off the column of step d); and f) contacting the uscfDNA sample from e) with Solid Phase Reversible Immobilization (SPRI) magnetic beads to clean up the uscfDNA.

In some embodiments, the method further comprises the step of preparing a sequencing library from the uscfDNA sample from step f). In some embodiments, the method further comprises the step of sequence the library of uscfDNA.

In some embodiments, the method further comprises contacting the eluted mncfDNA of step a) with SPRI magnetic beads to clean up the mncfDNA. In some embodiments, the method further comprises the step of preparing a sequencing library from the extracted mncfDNA. In some embodiments, the method further comprises the step of sequencing the library of mncfDNA.

In some embodiments, step b) is performed two or more times.

In some embodiments, the method further comprises a step of lysing a cell or disrupting proteins prior to step a). In some embodiments, the step of lysing a cell or disrupting proteins comprises: i) adding proteinase K and SDS to the sample; ii) incubating the sample for 30 minutes at 60 °C; and iii) cooling the sample to ambient room temperature.

In some embodiments, the method further comprises a step of isolating genomic DNA from the sample prior to step a).

In some embodiments, the sample is a biological fluid sample. In some embodiments, the sample is selected from the group consisting of a blood sample, a plasma sample, a saliva sample, a sputum sample, a urine sample, and a liquid biopsy sample.

In some embodiments, the invention provides a method of identifying novel biomarkers for diseases or disorders comprising obtaining mncfDNA, uscfDNA, or a combination thereof, from a sample according to the method of any one of claims 1-12 and analyzing the amount or sequence content of the mncfDNA, uscfDNA, or the combination thereof, to identify novel biomarkers of a disease or disorder. In some embodiments, the biomarker is selected from the group consisting of a mutation, an indel, a copy number variation, and a methylation marker. In some embodiments, the biomarker is an increase or decrease in the total amount of mncfDNA, uscfDNA, or a combination thereof in a test sample as compared to a control sample. In some embodiments, the biomarker is an increase or decrease in the amount of mncfDNA, uscfDNA, or a combination thereof associated with a specific gene in a test sample as compared to a control sample.

In some embodiments, the invention provides a method of diagnosing a diseases or disorder in a subject in need thereof, the method comprising obtaining a sample from the subject, isolating mncfDNA, uscfDNA, or a combination thereof from the sample according to the method of any one of claims 1-12; analyzing the amount or sequence content of the mncfDNA, uscfDNA, or a combination thereof to detect a biomarker of a disease or disorder, and diagnosing the subject as having or at risk of the disease or disorder associated with the identified biomarker. In some embodiments, the biomarker is selected from the group consisting of a mutation, an indel, a copy number variation, and a methylation marker. In some embodiments, the biomarker is an increase or decrease in the total amount of mncfDNA, uscfDNA, or a combination thereof in a test sample as compared to a control sample. In some embodiments, the biomarker is an increase or decrease in the amount of mncfDNA, uscfDNA, or a combination thereof associated with a specific gene in a test sample as compared to a control sample. In some embodiments, the disease or disorder is selected from the group consisting of an autoimmune disease or disorder, a disease or disorder associated with an infectious agent, and cancer.

In some embodiments, the invention provides a kit comprising components for performing a method of separately isolating mono-nucleosomal cfDNA (mncfDNA) and ultrashort single-stranded cell-free DNA (uscfDNA) molecules from a mixed nucleic acid sample.

### BRIEF DESCRIPTION OF THE DRAWINGS

FIG. 1 depicts representative schematic diagrams of the uscfDNA-seq protocol, demonstrating that classic QiaC extraction protocol yields only mncfDNA (120-200 bp) while the modified miRNA protocol QiaM leads to the retention of both mncfDNA and uscfDNA (25-100 bp).

FIG. 2, comprising FIG. 2A and FIG. 2B, depicts representative physical separation of ultrashort single stranded cell-free DNA in biofluids using differential extraction. FIG. 2A depicts a schematic representation of the differential filtration procedure. The initial sample is prepared witih QiaC reagents and passed through a silica column. The flowthrough is then transferred to a second silica column. The flowthrough of the second column is combined with QiaM reagents and passed through a third silica column. The elutions of each filter will contain different populations of cell-free DNA. FIG. 2B depicts representative imaging of an electrophoresis gel showing that QiaM only retains both mncfDNA (upper box) and ucsfDNA (lower box). QiaC₁ retains the majority of mncfDNA whereas QiaC₂ retains traces of ucsfDNA. The majority of uscfDNA is captured by and eluted from the QiaM₁ step. Fragments appear ~160 bp longer due to attachment of adapters.

FIG. 3, comprising FIG. 3A through FIG. 3C, depicts representative differential extraction using larger capacity silica filters. FIG. 3A depicts representative images of Qiagen vacuum-based filters for circulating nucleic acid isolation (left) and Zymogen high-capacity centrifuge-based silica filters. FIG. 3B depicts a schematic representation of the isolation process using larger capacity filters (Zymogen Quick-cfDNA Serum and Plasma Kit) which can work with up to 10 mL of sample for easier processing of large samples. FIG. 3C depicts representative imaging of an electrophoresis gel showing that, similar to the miniprep results in FIG. 2B, use of Zymo columns with QiaM only retains both mncfDNA (upper box) and ucsfDNA (middle box). Also as seen before, the first filtration with QiaC retained only mncfDNA. The second filtration with QiaC demonstrates capture of residual mncfDNA not captured in the first filtration and the third filtration, with QiaM, demonstrates capture of only uscfDNA.

FIG. 4 depicts representative sequencing results of libraries prepared from differentially extracted cfDNA. Sequenced libraries from QiaM-only elution demonstrate that the sample contains both uscfDNA (25-100 bp) and mncfDNA (120-200 bp). QiaC₁ elution retained only mncfDNA while the QiaM₁ elution retained only ucsfDNA.

FIG. 5 depicts a Next-generation Sequencing (NGS) pipeline to separate and detect ultrashort single-stranded cell-free DNA (uscfDNA).

### DETAILED DESCRIPTION

The invention is based, in part, on the development of a novel method for separating and isolating different DNA species from a single sample including genomic DNA, mononucleosomal cfDNA (mncfDNA) and ultrashort single-stranded cell-free DNA (uscfDNA). In some embodiments, the invention relates to sequencing libraries generated from samples containing or retaining uscfDNA, wherein the sequencing libraries have better coverage of promote and exon regions due to the presence of uscfDNA.

In some embodiments, the invention provides methods of use of samples in which the uscfDNA has been enriched for identification of novel biomarkers or for diagnosing diseases or disorders based on the detection of known biomarkers associated with diseases or disorders.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, an “adaptor” of the present invention means a piece of nucleic acid that is added to a nucleic acid of interest, e.g., the polynucleotide. Two adaptors of the present invention are preferably ligated to the ends of a DNA fragment cross-linked to a polypeptide of interest, with one adaptor on each end of the fragment. Adaptors of the present invention can comprise a primer binding sequence, a random nucleotide sequence, a barcode, or any combination thereof.

An affinity label, as the term us used herein, refers to a moiety that specifically binds another moiety and can be used to isolate or purify the affinity label, and compositions to which it is bound, from a complex mixture. One example of such an affinity label is a member of a specific binding pair (e.g, biotin: avidin, antibody: antigen). The use of affinity labels such as digoxigenin, dinitrophenol or fluorescein, as well as antigenic peptide ‘tags’ such as polyhistidine, FLAG, HA and Myc tags, is envisioned.

“Amplification,” as used herein, refers to any in vitro process for increasing the number of copies of a nucleotide sequence or sequences, i.e., creating an amplification product which may include, by way of example additional target molecules, or target-like molecules or molecules complementary to the target molecule, which molecules are created by virtue of the presence of the target molecule in the sample. These amplification processes include but are not limited to polymerase chain reaction (PCR), multiplex PCR, Rolling Circle PCR, ligase chain reaction (LCR) and the like, in a situation where the target is a nucleic acid, an amplification product can be made enzymatically with DNA or RNA polymerases or transcriptases. Nucleic acid amplification results in the incorporation of nucleotides into DNA or RNA. As used herein, one amplification reaction may consist of many rounds of DNA replication. PCR is an example of a suitable method for DNA amplification. For example, one PCR reaction may consist of 2-40 “cycles” of denaturation and replication.

“Amplification products,” “amplified products” “PCR products” or “amplicons” comprise copies of the target sequence and are generated by hybridization and extension of an amplification primer. This term refers to both single stranded and double stranded amplification primer extension products which contain a copy of the original target sequence, including intermediates of the amplification reaction.

A “barcode”, as used herein, refers to a nucleotide sequence that serves as a means of identification for sequenced polynucleotides of the present invention. Barcodes of the present invention may comprise at least 4, 5, 6, 7, 8, 9, 10, 11, 12, 13, 14, 15, 16, 17, 18, 19, 20 or more bases in length.

“Nucleic acid” or “oligonucleotide” or “polynucleotide” or “nucleic acid fragment” as used herein may mean at least two nucleotides covalently linked together. The depiction of a single strand also defines the sequence of the complementary strand, or the sequence of a molecule that hybridizes to at least a portion of the single strand sequence. Thus, a nucleic acid also encompasses the complementary strand of a depicted single strand as well as probes, primers or oligonucleotide sequences having complementarity to at least a portion of the strand. Many variants of a nucleic acid may be used for the same purpose as a given nucleic acid. Thus, a nucleic acid also encompasses substantially identical nucleic acids and complements thereof. A single strand provides a probe that may hybridize to a target sequence. Thus, a nucleic acid also encompasses a probe that hybridizes under appropriate hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo-and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. As used herein, the term nucleic acids includes both natural and non-natural nucleic acids. Non-natural nucleic acids include, but are not limited to, 2′F, 2′-fluoro; 2′OMe, 2′-O-methyl; LNA, locked nucleic acid; FANA, 2′-fluoro arabinose nucleic acid; HNA, hexitol nucleic acid; 2′MOE, 2′-O-methoxyethyl; ribuloNA, (1′-3′)-β-L-ribulo nucleic acid; TNA, α-L-threose nucleic acid; tPhoNA, 3′-2′ phosphonomethyl-threosyl nucleic acid; dXNA, 2′-deoxyxylonucleic acid; PS, phosphorothioate; phNA, alkyl phosphonate nucleic acid; and PNA, peptide nucleic acid.

“Primer” as used herein refers to a single-stranded oligonucleotide or a single-stranded polynucleotide that is extended on its 3′ end by covalent addition of nucleotide monomers during amplification. Nucleic acid amplification often is based on nucleic acid synthesis by a nucleic acid polymerase. Many such polymerases require the presence of a primer that can be extended to initiate such nucleic acid synthesis.

As used herein, “sample” or “test sample,” may refer to any source used to obtain nucleic acids for examination using the compositions and methods of the invention. A test sample is typically anything suspected of containing a target sequence.

Any DNA sample may be used in practicing the present invention, including without limitation eukaryotic, prokaryotic, viral DNA, non-natural DNA, cDNA, and recombinant DNA molecules.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention provides methods of capturing ultrashort nucleic acid molecules, methods of use thereof for sequencing library construction, and methods of use thereof to identify the quantity or sequence(s) of ultrashort cell free (uscf) nucleic acid molecules in a sample. In some embodiments, the uscf nucleic acid molecules are single stranded DNA molecules.

The present technology provides improved nucleic acid preparation methods suitable for enrichment, isolation, and analysis of ultrashort single stranded nucleic acid species sometimes found in cell free or substantially cell free biological compositions containing mixed compositions, and often associated with various disease conditions or apoptotic cellular events (e.g., cancers and cell proliferative disorders, prenatal or neonatal diseases, genetic abnormalities, and programmed cell death events). The ultrashort single stranded nucleic acid species targets, which can represent degraded or fractionated nucleic acids, can also be used for haplotyping and genotyping analysis, such as fetal genotyping for example.

Methods and compositions described herein are useful for size selection of ultrashort single-stranded cell-free DNA, in a simple, cost-effective manner that also can be compatible with automated and high throughput processes and apparatus. Methods provided herein are useful for enriching or extracting a target nucleic acid from a cell free or substantially cell free biological composition containing a mixture of non-target nucleic acids, based on the size of the nucleic acid, where the target nucleic acid is of a different size, and often is smaller, than the non-target nucleic acid.

### Methods for Obtaining and Using mncfDNA and uscfDNA

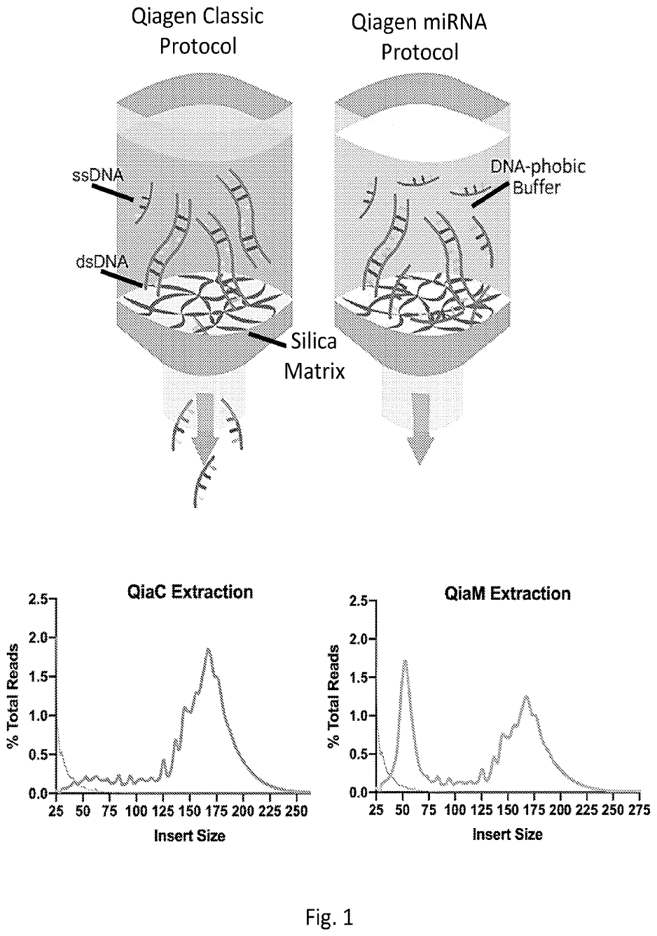

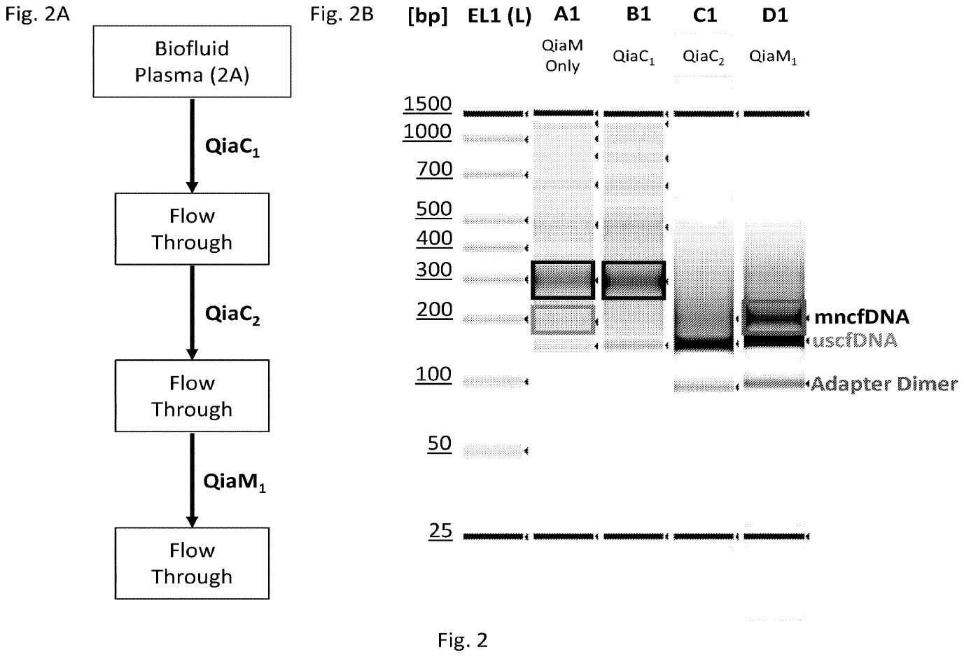

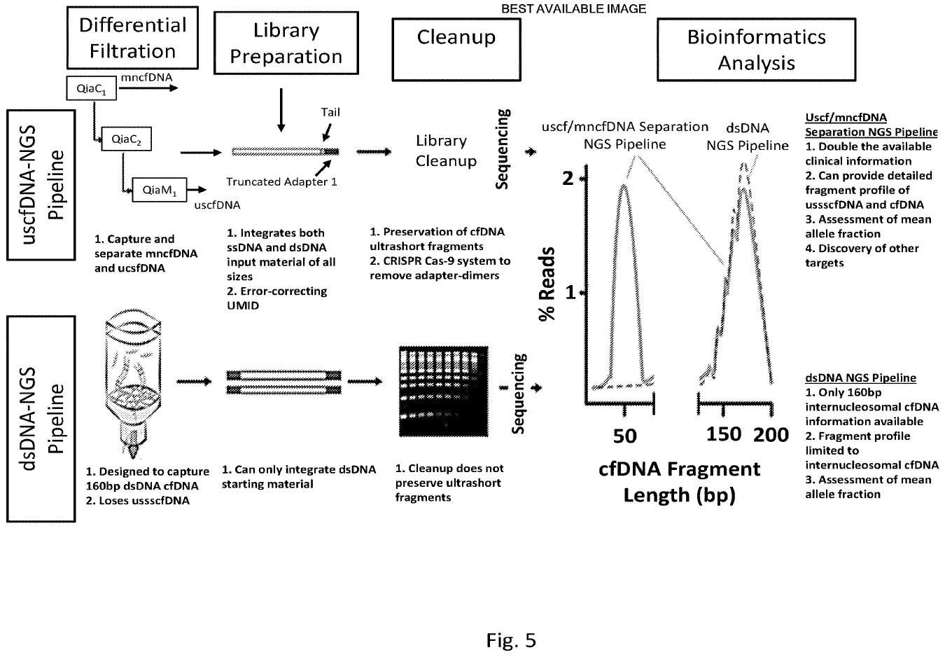

The invention is based, in part on the development of a new pipeline for isolating, enriching and sequencing mncfDNA and uscfDNA. It is represented in FIG. 1A, FIG. 2A, and FIG. 5. While the process is described for sequencing mncfDNA and uscfDNA from plasma samples, many of the process steps apply in sequencing mncfDNA and uscfDNA found in other types of sample such as urine, sweat, saliva etc. The baseline process may have the following steps: 1) collect a patient sample 2) extract mncfDNA from the sample using an extraction method optimized for mncfDNA 3) extract uscfDNA from the flowthrough sample using an extraction method optimized for uscfDNA, 4) prepare a sequencing library from the extracted uscfDNA and/or mncfDNA and 5) perform next generation sequencing on the sequencing library. However, the invention should no be limited to methods that require sequencing library preparation and sequencing as the isolated mncfDNA and uscfDNA can be utilized in any appropriate downstream assay or analysis including, but not limited to, binding assays, and immunoassays.

In some embodiments, the methods of the invention include a step of obtaining a plasma fraction of the whole blood sample, wherein the plasma fraction comprises the mncfDNA and/or uscfDNA. In some embodiments, the methods of the invention include a step of obtaining saliva sample wherein the saliva sample comprises the mncfDNA and/or uscfDNA.

In some embodiments, the invention relates to a method of isolating uscfDNA from a sample using the miRNA protocol of the QIAamp Circulating Nucleic Acid Kit, referred to herein as the QiaM method. In some embodiments, the invention relates to a method of isolating mncfDNA from a sample using the RNA protocol of the QIAamp Circulating Nucleic Acid Kit, referred to herein as the QiaC method.

In some embodiments, the invention relates to a method of separating uscfDNA and mncfDNA from a sample. In some embodiments, the method comprises a combination of QiaM and QiaC methods. In some embodiments, the method comprises isolating mncfDNA from a sample through a QiaC method and subsequently isolating uscfDNA from the sample through a QiaM method.

In some embodiments, the method comprises treating a sample with one or more buffers according to the QiaC method and filtering the solution to adsorb mncfDNA onto the filter. In some embodiments, the flowthrough resulting from the filtration is combined with one or more low-molecular weight alcohols and filtered to adsorb uscfDNA onto the filter. Examples of suitable low-molecular weight alcohols include, but are not limited to, propanol, isopropanol, n-butanol, iso-butanol, sec-butanol, tert-butanol, n-pentanol, 2-pentanol, 3-pentanol, 3-methyl-1-butanol, 2-methyl-1-butanol, 1-methyl-1-butanol, 2,2-dimethyl-1-propanol, 3-methyl-2-butanol, and 2-methyl-2-butanol. In some embodiments, the low-molecular weight alcohol is isopropanol.

In some embodiments, the filter comprises one or more substrates selected from the group consisting of silica, alumina, hydroxyapatite, poly[(2-methacryloyloxy)ethyl] trimethylammonium (poly(MATMA)), poly(glycerol 1,3-diglycerolate diacrylate) ((polyGDGDA), and poly(3-trimethoxysilylpropyl methacrylate) (poly(TMSPM)). In some embodiments, the one or more substrates are activated and/or modified with one or more positively charged moieties or dopants. Examples of compounds useful in modifying substrates include, but are not limited to, aminoalkyl silanes, alkanolamines, and iron (III) salts.

In some embodiments, the flowthrough resulting from the QiaC filtration is filtered through one or more additional filters prior to the addition of the low-molecular weight alcohol to adsorb remaining mncfDNA to the filters and produce an essentially mncfDNA-free flowthrough.

In some embodiments, the mncfDNA is eluted from the first filter to obtain a sample of mncfDNA. In some embodiments, the uscfDNA is eluted from the last filter to obtain a sample of uscfDNA.

### Library Preparation

In some embodiments the methods of the invention include the preparation of a sequencing library from the uscfDNA. In some embodiments, the method of the invention includes attaching sequencing adapters to ends of ultrashort single-stranded cell-free DNA fragments, thereby preparing a sequencing library comprising library fragments having the sequencing adapters attached to either end of the ultrashort single-stranded cell-free DNA fragments.

In some embodiments, a low molecular weight retention protocol for preparation of a sequencing library is followed for all bead-clean up steps during sequencing library preparation. In some embodiments, for double-stranded DNA libraries extracted uscfDNA is ligated to adapters using standard methodologies in the art with some modifications: the second (or post-PCR) purification is performed using 60 μl of purification beads in order to retain the uscfDNA fragments. In some embodiments, for double-stranded DNA libraries extracted uscfDNA is used as input and heat-shocked prior to ligation to adapters using a single-stranded library preparation method.

### Multiplex Sequencing

The large number of sequence reads that can be obtained per sequencing run permits the analysis of pooled samples i.e. multiplexing, which maximizes sequencing capacity and reduces workflow. For example, the massively parallel sequencing of eight libraries performed using the eight lane flow cell of the Illumina Genome Analyzer, and Illumina's HiSeq Systems, can be multiplexed to sequence two or more samples in each lane such that 16, 24, 32 etc. or more samples can be sequenced in a single run. Parallelizing sequencing for multiple samples i.e. multiplex sequencing, requires the incorporation of sample-specific index sequences, also known as barcodes, during the preparation of sequencing libraries. Sequencing indexes are distinct base sequences of about 5, about 10, about 15, about 20 about 25, or more bases that are added at the 3′ end of the genomic and marker nucleic acid. The multiplexing system enables sequencing of hundreds of biological samples within a single sequencing run. The preparation of indexed sequencing libraries for sequencing of clonally amplified sequences can be performed by incorporating an index sequence into a PCR primer used for cluster amplification. Alternatively, the index sequence can be incorporated into the adaptor, which is ligated to the uscfDNA prior to the PCR amplification. Sequencing of the uniquely marked indexed nucleic acids provides index sequence information that identifies samples in the pooled sample libraries, and sequence information of marker molecules correlates sequencing information of the genomic nucleic acids to the sample source. In embodiments wherein the multiple samples are sequenced individually i.e. singleplex sequencing, marker and uscfDNA of each sample need only be modified to contain the adaptor sequences as required by the sequencing platform and exclude the indexing sequences.

### Samples

In some embodiments, the sample containing uscfDNA is derived from a biological fluid, cell, tissue, organ, or organism, comprising a nucleic acid or a mixture of nucleic acids comprising at least one uscfDNA molecule. Such samples include, but are not limited to sputum/oral fluid, amniotic fluid, blood, a blood fraction, or fine needle biopsy samples (e.g., surgical biopsy, fine needle biopsy, etc.) urine, peritoneal fluid, pleural fluid, and the like. Although the sample is often taken from a human subject (e.g., patient), the assays can be from any mammal, including, but not limited to, dogs, cats, horses, goats, sheep, cattle, pigs, etc.

The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the uscf nucleic acid(s) of interest remain in the test sample. Such “treated” or “processed” samples are still considered to be biological samples with respect to the methods described herein.

### Applications

Sequence information generated as described herein can be used for any number of applications. Exemplary applications include, but are not limited to, determining mutations, indels, copy number variations (CNVs), identify methylation markers, or identifying biomarkers for diseases or disorders using the uscfDNA. The methods and apparatus described herein may employ next generation sequencing technology (NGS) as described elsewhere herein. In certain embodiments, clonally amplified uscfDNA molecules are sequenced in a massively parallel fashion within a flow cell (e.g. as described in Volkerding et al., 2009, Clin Chem, 55:641-658; Metzker, 2010, Nature Rev, 11:31-46). In addition to high-throughput sequence information, NGS provides quantitative information, in that each sequence read is a countable “sequence tag” representing an individual clonal DNA template or a single DNA molecule. In some embodiments, the methods and apparatus disclosed herein may employ the following some or all of the operations from the following: obtain a nucleic acid test sample 0.5 from a patient (typically by a non-invasive procedure); process the test sample in preparation for sequencing; sequence nucleic acids from the test sample to produce numerous reads (e.g., at least 10,000); align the reads to portions of a reference sequence/genome and determine the amount of DNA (e.g., the number of reads) that map to defined portions the reference sequence (e.g., to defined chromosomes or chromosome segments); calculate a dose of one or o more of the defined portions by normalizing the amount of DNA mapping to the defined portions with an amount of DNA mapping to one or more normalizing chromosomes or chromosome segments selected for the defined portion; determining whether the dose indicates that the defined portion is “affected” (e.g., aneuploidy or mosaic); reporting the determination and optionally converting it to a diagnosis; using the diagnosis or determination to develop a plan of treatment, monitoring, or further testing for the patient. In some embodiments, the biological sample is obtained from a subject and comprises a mixture of nucleic acids contributed by different subjects.

### Diagnostic Assays

In some embodiments, use of the methods described herein in the diagnosis, and/or monitoring, and or treating pathologies is contemplated. For example, the methods can be applied to determining the presence or absence of a disease, to monitoring the progression of a disease and/or the efficacy of a treatment regimen, or to determining the presence or absence of nucleic acids of a pathogen e.g. virus. To date a number of studies have reported biomarkers in genes involved in inflammation and the immune response, infectious disease, neurological and psychiatric diseases, and cancer. Biomarkers associated with these diseases and disorder can be identified in uscfDNA enriched samples generated according to the methods of the invention.

In some embodiments, blood, plasma and serum DNA from cancer patients contains measurable quantities of tumor DNA, that can be identified using the methods of the invention to identify the type or stage of the tumor. Identification of genomic instabilities associated with cancers that can be determined in the circulating uscfDNA in cancer patients is a potential diagnostic and prognostic tool. In one embodiment, methods described herein are used to determine a biomarker, mutation or CNV of one or more sequence(s) of interest in a sample, e.g., a sample comprising a mixture of nucleic acids derived from a subject that is suspected or is known to have cancer. In one embodiment, the sample is a plasma sample derived (processed) from peripheral blood that may comprise a mixture of uscfDNA derived from normal and cancerous cells.

In some embodiments, blood, plasma and serum DNA from a subject with a disease or disorder (e.g., an auto-immune disease or disorder) contains activated or inactivated genes due to differences in methylation, that can be identified using the methods of the invention.

Identification of biomarkers associated with diseases and disorders that can be determined in the circulating uscfDNA in patients is a potential diagnostic and prognostic tool. In one embodiment, methods described herein are used to determine novel biomarkers, mutations or CNVs for diseases or disorders.

### Data Processing

After isolating mncfDNA and/or uscfDNA as described herein, the mncfDNA and/or uscfDNA may be detected and/or analyzed by any suitable method and any suitable detection device. One or more target nucleic acids in the mncfDNA and/or uscfDNA may be detected and/or analyzed. In some embodiments, the uscfDNA may potentially contain somatic mutations or novel mutations useful for identifying cancer. In some embodiments, the mncfDNA and/or uscfDNA may contain methylated markers that can be used to identify auto-immunity diseases. In some embodiments, the mncfDNA and/or uscfDNA may also be useful for as a global biomarker in which its increase concentration may be diagnostic of aberrations in the patient's condition. Therefore, in some embodiments, the invention includes methods of diagnosing subjects based on the identification of a biomarker in mncfDNA and/or uscfDNA isolated according to the isolation methods of the invention.

In some embodiments, a diagnosis or the presence or absence of an outcome can be determined from the detection and/or analysis results. In some embodiments, the term “outcome” as used herein can refer to the presence, absence or total amount of one or more mncfDNA and/or uscfDNA nucleic acids in the sample. In some embodiments, the term “outcome” as used herein can refer to the presence, absence or amount of a biomarker in a population of mncfDNA and/or uscfDNA nucleic acids in the sample. In some embodiments, the term “outcome” as used herein can refer to an increase or decrease in the proportion of total mncfDNA and/or uscfDNA nucleic acids in the sample. In some embodiments, the term “outcome” as used herein can refer to identification of a disease, disorder or condition associated with the presence, absence, biomarker or total amount of one or more mncfDNA and/or uscfDNA nucleic acids in the sample. Non-limiting examples of outcomes include presence or absence of a fetus (e.g., a pregnancy test), prenatal or neonatal disorder, chromosome abnormality, chromosome aneuploidy (e.g., trisomy 21, trisomy 18, trisomy 13), a cellular proliferation condition (e.g., cancer), a cellular instability condition, an autoimmune disease or disorder and the like.

As described herein, algorithms, software, processors and/or machines, for example, can be utilized to (i) process detection data pertaining to mncfDNA and/or uscfDNA nucleic acid, and/or (ii) identify the presence or absence of an outcome.

The presence or absence of an outcome may be determined for all samples tested, or in some embodiments, the presence or absence of an outcome is determined in a subset of the samples (e.g., samples from individual subjects). An outcome may be determined for about 60, 65, 70, 75, 76, 77, 78, 79, 80, 81, 82, 83, 84, 85, 86, 87, 88, 89, 90, 91, 92, 93, 94, 95, 96, 97, 98, 99%, or greater than 99%, of samples analyzed in a set. A set of samples can include any suitable number of samples, and in some embodiments, a set has about 10, 15, 20, 25, 30, 35, 40, 45, 50, 55, 60, 65, 70, 75, 80, 90, 100, 200, 300, 400, 500, 600, 700, 800, 900 or 1000 samples, or more than 1000 samples. The set may be considered with respect to samples tested in a particular period of time, and/or at a particular location. The set may be otherwise defined by, for example, age and/or ethnicity. The set may be comprised of a sample which is subdivided into subsamples or replicates all or some of which may be tested. The set may comprise a sample from the same subject collected at two different times. An outcome may be determined about 60% or more of the time for a given sample analyzed (e.g., about 65, 70, 75, 76, 77, 78, 79, 80, 81, 82, 83, 84, 85, 86, 87, 88, 89, 90, 91, 92, 93, 94, 95, 96, 97, 98, 99%, or more than 99% of the time for a given sample). Analyzing a higher number of characteristics (e.g., sequence variations) that discriminate alleles can increase the percentage of outcomes determined for the samples (e.g., discriminated in a multiplex analysis). One or more fluid samples (e.g., one or more blood samples) may be provided by a subject. One or more mncfDNA and/or uscfDNA enriched samples, or two or more replicate mncfDNA and/or uscfDNA enriched samples, may be isolated from a single fluid sample, and analyzed by methods described herein.

Presence or absence of an outcome can be expressed in any suitable form, and in conjunction with any suitable variable, collectively including, without limitation, ratio, deviation in ratio, frequency, distribution, probability (e.g., odds ratio, p-value), likelihood, percentage, value over a threshold, or risk factor, associated with the presence of a outcome for a subject or sample. An outcome may be provided with one or more variables, including, but not limited to, sensitivity, specificity, standard deviation, probability, ratio, coefficient of variation (CV), threshold, score, probability, confidence level, or combination of the foregoing, in certain embodiments.

One or more of ratio, sensitivity, specificity and/or confidence level may be expressed as a percentage. The percentage, independently for each variable, may be greater than about 90% (e.g., about 90, 91, 92, 93, 94, 95, 96, 97, 98 or 99%, or greater than 99% (e.g., about 99.5%, or greater, about 99.9% or greater, about 99.95% or greater, about 99.99% or greater)). Coefficient of variation (CV) in some embodiments is expressed as a percentage, and sometimes the percentage is about 10% or less (e.g., about 10, 9, 8, 7, 6, 5, 4, 3, 2 or 1%, or less than 1% (e.g., about 0.5% or less, about 0.1% or less, about 0.05% or less, about 0.01% or less)). A probability (e.g., that a particular outcome determined by an algorithm is not due to chance) in certain embodiments is expressed as a p-value, and sometimes the p-value is about 0.05 or less (e.g., about 0.05, 0.04, 0.03, 0.02 or 0.01, or less than 0.01 (e.g., about 0.001 or less, about 0.0001 or less, about 0.00001 or less, about 0.000001 or less)).

For example, scoring or a score may refer to calculating the probability that a particular outcome is actually present or absent in a subject/sample. The value of a score may be used to determine for example the variation, difference, or ratio of amplified nucleic detectable product that may correspond to the actual outcome. For example, calculating a positive score from detectable products can lead to an identification of an outcome, which is particularly relevant to analysis of single samples.

Simulated (or simulation) data can aid data processing for example by training an algorithm or testing an algorithm. Simulated data may for instance involve hypothetical various samples of different concentrations of uscfDNA in serum, plasma, saliva and the like. Simulated data may be based on what might be expected from a real population or may be skewed to test an algorithm and/or to assign a correct classification based on a simulated data set. Simulated data also is referred to herein as “virtual” data. Simulations can be performed in most instances by a computer program. One possible step in using a simulated data set is to evaluate the confidence of the identified results, i.e. how well the selected positives/negatives match the sample and whether there are additional variations. A common approach is to calculate the probability value (p-value) which estimates the probability of a random sample having better score than the selected one. As p-value calculations can be prohibitive in certain circumstances, an empirical model may be assessed, in which it is assumed that at least one sample matches a reference sample (with or without resolved variations). Alternatively other distributions such as Poisson distribution can be used to describe the probability distribution.

An algorithm can assign a confidence value to the true positives, true negatives, false positives and false negatives calculated. The assignment of a likelihood of the occurrence of a outcome can also be based on a certain probability model.

Simulated data often is generated in an in silico process. As used herein, the term “in silico” refers to research and experiments performed using a computer. In silico methods include, but are not limited to, molecular modeling studies, karyotyping, genetic calculations, biomolecular docking experiments, and virtual representations of molecular structures and/or processes, such as molecular interactions.

As used herein, a “data processing routine” refers to a process that can be embodied in software that determines the biological significance of acquired data (i.e., the ultimate results of an assay). For example, a data processing routine can determine the amount of each nucleotide sequence species based upon the data collected. A data processing routine also may control an instrument and/or a data collection routine based upon results determined. A data processing routine and a data collection routine often are integrated and provide feedback to operate data acquisition by the instrument, and hence provide assay-based judging methods provided herein.

As used herein, software refers to computer readable program instructions that, when executed by a computer, perform computer operations. Typically, software is provided on a program product containing program instructions recorded on a computer readable medium, including, but not limited to, magnetic media including floppy disks, hard disks, and magnetic tape; and optical media including CD-ROM discs, DVD discs, magneto-optical discs, and other such media on which the program instructions can be recorded.

Different methods of predicting abnormality or normality can produce different types of results. For any given prediction, there are four possible types of outcomes: true positive, true negative, false positive or false negative. The term “true positive” as used herein refers to a subject correctly diagnosed as having a outcome. The term “false positive” as used herein refers to a subject wrongly identified as having a outcome. The term “true negative” as used herein refers to a subject correctly identified as not having a outcome. The term “false negative” as used herein refers to a subject wrongly identified as not having a outcome. Two measures of performance for any given method can be calculated based on the ratios of these occurrences: (i) a sensitivity value, the fraction of predicted positives that are correctly identified as being positives (e.g., the fraction of nucleotide sequence sets correctly identified by level comparison detection/determination as indicative of outcome, relative to all nucleotide sequence sets identified as such, correctly or incorrectly), thereby reflecting the accuracy of the results in detecting the outcome; and (ii) a specificity value, the fraction of predicted negatives correctly identified as being negative (the fraction of nucleotide sequence sets correctly identified by level comparison detection/determination as indicative of chromosomal normality, relative to all nucleotide sequence sets identified as such, correctly or incorrectly), thereby reflecting accuracy of the results in detecting the outcome.

The term “sensitivity” as used herein refers to the number of true positives divided by the number of true positives plus the number of false negatives, where sensitivity (sens) may be within the range of 0≤sens≤1. Ideally, method embodiments herein have the number of false negatives equaling zero or close to equaling zero, so that no subject is wrongly identified as not having at least one outcome when they indeed have at least one outcome. Conversely, an assessment often is made of the ability of a prediction algorithm to classify negatives correctly, a complementary measurement to sensitivity. The term “specificity” as used herein refers to the number of true negatives divided by the number of true negatives plus the number of false positives, where sensitivity (spec) may be within the range of 0≤spec≤1. Ideally, methods embodiments herein have the number of false positives equaling zero or close to equaling zero, so that no subject wrongly identified as having at least one outcome when they do not have the outcome being assessed. Hence, a method that has sensitivity and specificity equaling one, or 100%, sometimes is selected.

One or more prediction algorithms may be used to determine significance or give meaning to the detection data collected under variable conditions that may be weighed independently of or dependently on each other. The term “variable” as used herein refers to a factor, quantity, or function of an algorithm that has a value or set of values. For example, a variable may be the design of a set of amplified nucleic acid species, the number of sets of amplified nucleic acid species, type of outcome assayed, and the like.

Any suitable type of method or prediction algorithm may be utilized to give significance to the data of the present technology within an acceptable sensitivity and/or specificity. For example, prediction algorithms such as Mann-Whitney U Test, binomial test, log odds ratio, Chi-squared test, z-test, t-test, ANOVA (analysis of variance), regression analysis, neural nets, fuzzy logic, Hidden Markov Models, multiple model state estimation, and the like may be used. One or more methods or prediction algorithms may be determined to give significance to the data having different independent and/or dependent variables of the present technology. And one or more methods or prediction algorithms may be determined not to give significance to the data having different independent and/or dependent variables of the present technology. One may design or change parameters of the different variables of methods described herein based on results of one or more prediction algorithms (e.g., number of sets analyzed, types of nucleotide species in each set).

Several algorithms may be chosen to be tested. These algorithms then can be trained with raw data. For each new raw data sample, the trained algorithms will assign a classification to that sample (e.g., trisomy or normal). Based on the classifications of the new raw data samples, the trained algorithms' performance may be assessed based on sensitivity and specificity. Finally, an algorithm with the highest sensitivity and/or specificity or combination thereof may be identified.

Provided are methods for identifying the presence or absence of an outcome that comprise: (a) providing a system, wherein the system comprises distinct software modules, and wherein the distinct software modules comprise a signal detection module, a logic processing module, and a data display organization module; (b) detecting signal information indicating the presence, absence or amount of enriched nucleic acid; (c) receiving, by the logic processing module, the signal information; (d) calling the presence or absence of an outcome by the logic processing module; and (e) organizing, by the data display organization model in response to being called by the logic processing module, a data display indicating the presence or absence of the outcome.

Provided also are methods for identifying the presence or absence of an outcome, which comprise providing signal information indicating the presence, absence or amount of enriched nucleic acid; providing a system, wherein the system comprises distinct software modules, and wherein the distinct software modules comprise a signal detection module, a logic processing module, and a data display organization module; receiving, by the logic processing module, the signal information; calling the presence or absence of an outcome by the logic processing module; and, organizing, by the data display organization model in response to being called by the logic processing module, a data display indicating the presence or absence of the outcome.

Provided also are methods for identifying the presence or absence of an outcome, which comprise providing a system, wherein the system comprises distinct software modules, and wherein the distinct software modules comprise a signal detection module, a logic processing module, and a data display organization module; receiving, by the logic processing module, signal information indicating the presence, absence or amount of enriched nucleic acid; calling the presence or absence of an outcome by the logic processing module; and, organizing, by the data display organization model in response to being called by the logic processing module, a data display indicating the presence or absence of the outcome.

By “providing signal information” is meant any manner of providing the information, including, for example, computer communication means from a local, or remote site, human data entry, or any other method of transmitting signal information. The signal information may be generated in one location and provided to another location.

By “obtaining” or “receiving” signal information is meant receiving the signal information by computer communication means from a local, or remote site, human data entry, or any other method of receiving signal information. The signal information may be generated in the same location at which it is received, or it may be generated in a different location and transmitted to the receiving location.

By “indicating” or “representing” the amount is meant that the signal information is related to, or correlates with, for example, the amount of enriched nucleic acid or presence or absence of enriched nucleic acid. The information may be, for example, the calculated data associated with the presence or absence of enriched nucleic acid as obtained, for example, after converting raw data obtained by mass spectrometry.

Also provided are computer program products, such as, for example, a computer program products comprising a computer usable medium having a computer readable program code embodied therein, the computer readable program code adapted to be executed to implement a method for identifying the presence or absence of an outcome, which comprises (a) providing a system, wherein the system comprises distinct software modules, and wherein the distinct software modules comprise a signal detection module, a logic processing module, and a data display organization module; (b) detecting signal information indicating the presence, absence or amount of enriched nucleic acid; (c) receiving, by the logic processing module, the signal information; (d) calling the presence or absence of an outcome by the logic processing module; and, organizing, by the data display organization model in response to being called by the logic processing module, a data display indicating the presence or absence of the outcome.

Also provided are computer program products, such as, for example, computer program products comprising a computer usable medium having a computer readable program code embodied therein, the computer readable program code adapted to be executed to implement a method for identifying the presence or absence of an outcome, which comprises providing a system, wherein the system comprises distinct software modules, and wherein the distinct software modules comprise a signal detection module, a logic processing module, and a data display organization module; receiving signal information indicating the presence, absence or amount of enriched nucleic acid; calling the presence or absence of an outcome by the logic processing module; and, organizing, by the data display organization model in response to being called by the logic processing module, a data display indicating the presence or absence of the outcome.

Signal information may be, for example, mass spectrometry data obtained from mass spectrometry of uscfDNA, or of a uscfDNA enriched sample. As the uscfDNA may be amplified into a nucleic acid that is detected, the signal information may be detection information, such as mass spectrometry data, obtained from uscf nucleic acid or stoichiometrically amplified nucleic acid from the uscf nucleic acid, for example. The mass spectrometry data may be raw data, such as, for example, a set of numbers, or, for example, a two dimensional display of the mass spectrum. The signal information may be converted or transformed to any form of data that may be provided to, or received by, a computer system. The signal information may also, for example, be converted, or transformed to identification data or information representing an outcome. An outcome may be, for example, a fetal allelic ratio, or a particular chromosome number in fetal cells. Where the chromosome number is greater or less than in euploid cells, or where, for example, the chromosome number for one or more of the chromosomes, for example, 21, 18, or 13, is greater than the number of other chromosomes, the presence of a chromosomal disorder may be identified.

Also provided is a machine for identifying the presence or absence of an outcome wherein the machine comprises a computer system having distinct software modules, and wherein the distinct software modules comprise a signal detection module, a logic processing module, and a data display organization module, wherein the software modules are adapted to be executed to implement a method for identifying the presence or absence of an outcome, which comprises (a) detecting signal information indicating the presence, absence or amount of uscf nucleic acid; (b) receiving, by the logic processing module, the signal information; (c) calling the presence or absence of an outcome by the logic processing module, wherein a ratio of alleles different than a normal ratio is indicative of a chromosomal disorder; and (d) organizing, by the data display organization model in response to being called by the logic processing module, a data display indicating the presence or absence of the outcome. The machine may further comprise a memory module for storing signal information or data indicating the presence or absence of a chromosomal disorder. Also provided are methods for identifying the presence or absence of an outcome, wherein the methods comprise the use of a machine for identifying the presence or absence of an outcome.

Also provided are methods identifying the presence or absence of an outcome that comprises: (a) detecting signal information, wherein the signal information indicates presence, absence or amount of uscf nucleic acid; (b) transforming the signal information into identification data, wherein the identification data represents the presence or absence of the outcome, whereby the presence or absence of the outcome is identified based on the signal information; and (c) displaying the identification data.

Also provided are methods for identifying the presence or absence of an outcome that comprises:
(a) providing signal information indicating the presence, absence or amount of uscfDNA; (b) transforming the signal information representing into identification data, wherein the identification data represents the presence or absence of the outcome, whereby the presence or absence of the outcome is identified based on the signal information, and (c) displaying the identification data.

Also provided are methods for identifying the presence or absence of an outcome that comprises:
(a) receiving signal information indicating the presence, absence or amount of uscfDNA; (b) transforming the signal information into identification data, wherein the identification data represents the presence or absence of the outcome, whereby the presence or absence of the outcome is identified based on the signal information; and (c) displaying the identification data.

For purposes of these, and similar embodiments, the term “signal information” indicates information readable by any electronic media, including, for example, computers that represent data derived using the present methods. For example, “signal information” can represent the amount of uscf nucleic acid or amplified nucleic acid. Signal information, such as in these examples, that represents physical substances may be transformed into identification data, such as a visual display that represents other physical substances, such as, for example, a chromosome disorder, or a chromosome number. Identification data may be displayed in any appropriate manner, including, but not limited to, in a computer visual display, by encoding the identification data into computer readable media that may, for example, be transferred to another electronic device (e.g., electronic record), or by creating a hard copy of the display, such as a print out or physical record of information. The information may also be displayed by auditory signal or any other means of information communication. In some embodiments, the signal information may be detection data obtained using methods to detect uscf nucleic acid.

Once the signal information is detected, it may be forwarded to the logic-processing module. The logic-processing module may “call” or “identify” the presence or absence of an outcome.

Provided also are methods for transmitting genetic information to a subject, which comprise identifying the presence or absence of an outcome wherein the presence or absence of the outcome has been determined from determining the presence, absence or amount of uscf nucleic acid from a sample from the subject; and transmitting the presence or absence of the outcome to the subject. A method may include transmitting prenatal genetic information to a human pregnant female subject, and the outcome may be presence or absence of a chromosome abnormality or aneuploidy, in certain embodiments.

The term “identifying the presence or absence of an outcome” or “an increased risk of an outcome,” as used herein refers to any method for obtaining such information, including, without limitation, obtaining the information from a laboratory file. A laboratory file can be generated by a laboratory that carried out an assay to determine the presence or absence of an outcome. The laboratory may be in the same location or different location (e.g., in another country) as the personnel identifying the presence or absence of the outcome from the laboratory file. For example, the laboratory file can be generated in one location and transmitted to another location in which the information therein will be transmitted to the subject. The laboratory file may be in tangible form or electronic form (e.g., computer readable form), in certain embodiments.

The term “transmitting the presence or absence of the outcome to the subject” or any other information transmitted as used herein refers to communicating the information to the subject, or family member, guardian or designee thereof, in a suitable medium, including, without limitation, in verbal, document, or file form.

Also provided are methods for providing to a subject a medical prescription based on genetic information, which comprise identifying the presence or absence of an outcome, wherein the presence or absence of the outcome has been determined from the presence, absence or amount of uscf nucleic acid from a sample from the subject, and providing a medical prescription based on the presence or absence of the outcome to the subject.

The term “providing a medical prescription based on genetic information” refers to communicating the prescription to the subject, or family member, guardian or designee thereof, in a suitable medium, including, without limitation, in verbal, document or file form. The medical prescription may be for any course of action determined by, for example, a medical professional upon reviewing the uscf genetic information. For example, the medical prescription may be for the subject to undergo additional testing or confirmatory testing. In yet another example, the medical prescription may be medical advice to not undergo further testing.

Also provided are files, such as, for example, a file comprising the presence or absence of outcome for a subject, wherein the presence or absence of the outcome has been determined from the presence, absence or amount of uscf nucleic acid in a sample from the subject. The file may be, for example, but not limited to, a computer readable file, a paper file, or a medical record file.

Computer program products include, for example, any electronic storage medium that may be used to provide instructions to a computer, such as, for example, a removable storage device, CD-ROMS, a hard disk installed in hard disk drive, signals, magnetic tape, DVDs, optical disks, flash drives, RAM or floppy disk, and the like.

The systems discussed herein may further comprise general components of computer systems, such as, for example, network servers, laptop systems, desktop systems, handheld systems, personal digital assistants, computing kiosks, and the like. The computer system may comprise one or more input means such as a keyboard, touch screen, mouse, voice recognition or other means to allow the user to enter data into the system. The system may further comprise one or more output means such as a CRT or LCD display screen, speaker, FAX machine, impact printer, inkjet printer, black and white or color laser printer or other means of providing visual, auditory or hardcopy output of information.

The input and output means may be connected to a central processing unit which may comprise among other components, a microprocessor for executing program instructions and memory for storing program code and data. In some embodiments the methods may be implemented as a single user system located in a single geographical site. In other embodiments methods may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by the provider or it may be implemented as an Internet based service where the user accesses a web page to enter and retrieve information.

The various software modules associated with the implementation of the present products and methods can be suitably loaded into the computer system as desired, or the software code can be stored on a computer-readable medium such as a floppy disk, magnetic tape, or an optical disk, or the like. In an online implementation, a server and web site maintained by an organization can be configured to provide software downloads to remote users. As used herein, “module,” including grammatical variations thereof, means, a self-contained functional unit which is used with a larger system. For example, a software module is a part of a program that performs a particular task. Thus, provided herein is a machine comprising one or more software modules described herein, where the machine can be, but is not limited to, a computer (e.g., server) having a storage device such as floppy disk, magnetic tape, optical disk, random access memory and/or hard disk drive, for example.

The present methods may be implemented using hardware, software or a combination thereof and may be implemented in a computer system or other processing system. An example computer system may include one or more processors. A processor can be connected to a communication bus. The computer system may include a main memory, sometimes random access memory (RAM), and can also include a secondary memory. The secondary memory can include, for example, a hard disk drive and/or a removable storage drive, representing a floppy disk drive, a magnetic tape drive, an optical disk drive, memory card etc. The removable storage drive reads from and/or writes to a removable storage unit in a well-known manner. A removable storage unit includes, but is not limited to, a floppy disk, magnetic tape, optical disk, etc. which is read by and written to by, for example, a removable storage drive. As will be appreciated, the removable storage unit includes a computer usable storage medium having stored therein computer software and/or data.

Alternatively, secondary memory may include other similar means for allowing computer programs or other instructions to be loaded into a computer system. Such means can include, for example, a removable storage unit and an interface device. Examples of such can include a program cartridge and cartridge interface (such as that found in video game devices), a removable memory chip (such as an EPROM, or PROM) and associated socket, and other removable storage units and interfaces which allow software and data to be transferred from the removable storage unit to a computer system.

The computer system may also include a communications interface. A communications interface allows software and data to be transferred between the computer system and external devices. Examples of communications interface can include a modem, a network interface (such as an Ethernet card), a communications port, a PCMCIA slot and card, etc. Software and data transferred via communications interface are in the form of signals, which can be electronic, electromagnetic, optical or other signals capable of being received by communications interface. These signals are provided to communications interface via a channel. This channel carries signals and can be implemented using wire or cable, fiber optics, a phone line, a cellular phone link, an RF link and other communications channels. Thus, in one example, a communications interface may be used to receive signal information to be detected by the signal detection module.

In a related aspect, the signal information may be input by a variety of means, including but not limited to, manual input devices or direct data entry devices (DDEs). For example, manual devices may include, keyboards, concept keyboards, touch sensitive screens, light pens, mouse, tracker balls, joysticks, graphic tablets, scanners, digital cameras, video digitizers and voice recognition devices. DDEs may include, for example, bar code readers, magnetic strip codes, smart cards, magnetic ink character recognition, optical character recognition, optical mark recognition, and turnaround documents. In one embodiment, an output from a gene or chip reader my serve as an input signal.

### EFIRM Based Analysis of uscfDNA

In some embodiments, uscfDNA isolated according to the method of the invention can be applied to an EFIRM system for the detection of biomarkers. In some embodiments, the EFIRM assay includes a multiplexing electrochemical sensor for detecting biomarkers. The device utilizes a small sample volume with high accuracy. In addition, multiple markers can be measured simultaneously on the device with single sample loading. The device may significantly reduce the cost to the health care system, by decreasing the burden of patients returning to clinics and laboratories.

In one embodiment, the electrochemical sensor is an array of electrode chips (EZ Life Bio, USA). In one embodiment, each unit of the array has a working electrode, a counter electrode, and a reference electrode. The three electrodes may be constructed of bare gold or other conductive material before the reaction, such that the specimens may be immobilized on the working electrode. Electrochemical current can be measured between the working electrode and counter electrode under the potential between the working electrode and the reference electrode. The potential profile can be a constant value, a linear sweep, or a cyclic square wave, for example. An array of plastic wells may be used to separate each three-electrode set, which helps avoid the cross contamination between different sensors. In one embodiment, a three-electrode set is in each well of a 96 well gold electrode plate. A conducting polymer may also be deposited on the working electrodes as a supporting film, and in some embodiments, as a surface to functionalize the working electrode. As contemplated herein, any conductive polymer may be used, such as polypyrroles, polanilines, polyacetylenes, polyphenylenevinylenes, polythiophenes and the like.

In one embodiment, a cyclic square wave electric field is generated across the electrode within the sample well. In certain embodiments, the square wave electric field is generated to aid in polymerization of one or more capture probes to the polymer of the sensor. In certain embodiments, the square wave electric field is generated to aid in the hybridization of the capture probes with the marker and/or detector probe. The positive potential in the csw E-field helps the molecules accumulate onto the working electrode, while the negative potential removes the weak nonspecific binding, to generate enhanced specificity. Further, the flapping between positive and negative potential across the cyclic square wave also provides superior mixing during incubation, without disruption of the desired specific binding, which accelerates the binding process and results in a faster test or assay time. In one embodiment, a square wave cycle may consist of a longer low voltage period and a shorter high voltage period, to enhance binding partner hybridization within the sample. While there is no limitation to the actual time periods selected, examples include 0.15 to 60 second low voltage periods and 0.1 to 60 second high voltage periods. In one embodiment, each square-wave cycle consists of 1 s at low voltage and 1 s at high voltage. For hybridization, the low voltage may be around −200 mV and the high voltage may be around +500 mV. In some embodiments, the total number of square wave cycles may be between 2-50. In one embodiment, 5 cyclic square-waves are applied for each surface reaction. With the csw E-field, both the polymerization and hybridization are finished on the same chip within minutes. In some embodiments, the total detection time from sample loading is less than 30 minutes. In other embodiments, the total detection time from sample loading is less than 20 minutes. In other embodiments, the total detection time from sample loading is less than 10 minutes. In other embodiments, the total detection time from sample loading is less than 5 minutes. In other embodiments, the total detection time from sample loading is less than 2 minutes. In other embodiments, the total detection time from sample loading is less than 1 minute.

A multi-channel electrochemical reader (EZ Life Bio) controls the electrical field applied onto the array sensors and reports the amperometric current simultaneously. In practice, solutions can be loaded onto the entire area of the three-electrode region including the working, counter, and reference electrodes, which are confined and separated by the array of plastic wells. After each step, the electrochemical sensors can be rinsed with ultrapure water or other washing solution and then dried, such as under pure N2. In some embodiments, the sensors are single use, disposable sensors. In other embodiment, the sensors are reusable.

### Determining Effectiveness of Therapy or Prognosis

In one aspect, the level of one or more mncfDNA and/or uscfDNA, or a biomarker identified therein, in a biological sample of a patient is used to monitor the effectiveness of treatment or the prognosis of disease. In some embodiments, the level of one or more mncfDNA and/or uscfDNA, or a biomarker identified therein, in a test sample obtained from a treated patient can be compared to the level from a reference sample obtained from that patient before initiation of a treatment. Clinical monitoring of treatment typically entails that each patient serves as his or her own baseline control. In some embodiments, test samples are obtained at multiple time points following administration of the treatment. In these embodiments, measurement of the level of one or more mncfDNA and/or uscfDNA, or a biomarker identified therein, in the test samples provides an indication of the extent and duration of in vivo effect of the treatment.

Measurement of the level of one or more mncfDNA and/or uscfDNA, may allow for the course of treatment of a disease to be monitored. The effectiveness of a treatment regimen for a disease can be monitored by detecting one or more mncfDNA and/or uscfDNA in an effective amount from samples obtained from a subject over time and comparing the detected level of one or more mncfDNA and/or uscfDNA. For example, a first sample can be obtained before the subject receives treatment and one or more subsequent samples are taken after or during treatment of the subject. Changes in mncfDNA and/or uscfDNA levels across the samples may provide an indication as to the effectiveness of the therapy.

In some embodiments, the disclosure provides a method for monitoring the levels of mncfDNA and/or uscfDNA in response to treatment. For example, in certain embodiments, the disclosure provides for a method of determining the efficacy of treatment in a subject, by measuring the levels of one or more mncfDNA and/or uscfDNA as described herein. In some embodiments, the level of the one or more uscfDNA can be measured over time, where the level at one timepoint after the initiation of treatment is compared to the level at another timepoint after the initiation of treatment. In some embodiments, the level of the one or more mncfDNA and/or uscfDNA can be measured over time, where the level at one timepoint after the initiation of treatment is compared to the level before initiation of treatment.

In some embodiments, mncfDNA and/or uscfDNA levels can be used to identify therapeutics or drugs that are appropriate for a specific subject. For example, a test sample from the subject can be exposed to a therapeutic agent or a drug, and the level of one or more mncfDNA and/or uscfDNA can be determined. MncfDNA and/or uscfDNA levels can be compared to a sample derived from the subject before and after treatment or exposure to a therapeutic agent or a drug or can be compared to samples derived from one or more subjects who have shown improvements relative to a disease as a result of such treatment or exposure. Thus, in one aspect, the disclosure provides a method of assessing the efficacy of a therapy with respect to a subject comprising taking a first measurement of mncfDNA and/or uscfDNA or a mncfDNA and/or uscfDNA panel in a first sample from the subject; effecting the therapy with respect to the subject; taking a second measurement of the mncfDNA and/or uscfDNA or mncfDNA and/or uscfDNA panel in a second sample from the subject and comparing the first and second measurements to assess the efficacy of the therapy.

Accordingly, treatments or therapeutic regimens for use in can be selected based on the amounts of a specific mncfDNA and/or uscfDNA or a panel in samples obtained from the subjects and compared to a reference value. Two or more treatments or therapeutic regimens can be evaluated in parallel to determine which treatment or therapeutic regimen would be the most efficacious for use in a subject to delay onset, or slow progression of a disease. In various embodiments, a recommendation is made on whether to initiate or continue treatment of a disease.

A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example, prognosis can be expressed as a percent chance that a patient will survive after one year, five years, ten years or the like. Alternatively, prognosis may be expressed as the number of years, on average that a patient can expect to survive as a result of a condition or disease. The prognosis of a patient may be considered as an expression of relativism, with many factors affecting the ultimate outcome. For example, for patients with certain conditions, prognosis can be appropriately expressed as the likelihood that a condition may be treatable or curable, or the likelihood that a disease will go into remission, whereas for patients with more severe conditions, prognosis may be more appropriately expressed as likelihood of survival for a specified period of time. Additionally, a change in a clinical factor from a baseline level may impact a patient's prognosis, and the degree of change in level of the clinical factor may be related to the severity of adverse events. Statistical significance is often determined by comparing two or more populations and determining a confidence interval and/or a p value.

Multiple determinations of mncfDNA and/or uscfDNA levels can be made, and a temporal change in mncfDNA and/or uscfDNA level can be used to determine a prognosis. For example, comparative measurements are made of the mncfDNA and/or uscfDNAlevel in a patient at multiple time points, and a comparison of the mncfDNA and/or uscfDNA level at two or more time points may be indicative of a particular prognosis.

In certain embodiments, other prognostic factors may be combined with the mncfDNA and/or uscfDNA level or other biomarkers in the algorithm to determine prognosis with greater accuracy. Exemplary additional prognostic factors may include one or more prognostic factors selected from the group consisting of cytogenetics, performance status, age, gender and contemporary diagnosis.

### Treatments

In one aspect, the disclosure provides a method of diagnosing, treating or preventing a disease or disorder associated with a biomarker identified from analysis of mncfDNA and/or uscfDNA, an altered level of a specific mncfDNA and/or uscfDNA or a general increase or decrease of total mncfDNA and/or uscfDNA. In some embodiments, the method comprises administering to the subject an effective amount of a pharmaceutical agent for the treatment of a disease or disorder identified associated with a biomarker identified from analysis of mncfDNA and/or uscfDNA, an altered level of a specific mncfDNA and/or uscfDNA or a general increase or decrease of total mncfDNA and/or uscfDNA.

### Kits

The present invention further includes an assay kit containing the components for performing a mncfDNA and/or uscfDNA isolation assay of the invention, including, but not limited to, reagents, enzymes, buffers, separation beads, tubes, and instructions for the set-up, performance, monitoring, and interpretation of the assays of the present invention. Optionally, the kit may include control reagents and reagents for the detection of at least one biomarkers.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Physical Separation of DNA by Size for Isolation of Ultrashort Single-stranded Cell-free DNA

As all methods of isolating uscfDNA resulted in an impure combination of uscfDNA and mncfDNA, a method to separate these nucleic acids was developed in order to provide cleaner samples of each type of cfDNA. Noting that QiaC isolation yielded only mncfDNA and allowed ucsfDNA to freely flow through the filter while QiaM isolation yielded both mncfDNA and ucsfDNA, a pipeline for select isolation of each was developed using multi-filtration (FIG. 1). Samples were prepared according to normal QiaC protocol and filtered through a DNA silica column via centrifugation and the flowthrough collected in a 1.5 mL Eppendorf tube. The flowthrough was then filtered through a second DNA silica column and the flowthrough collected. QiaM reagents were then added to the resulting flowthrough, which was filtered with a third DNA silica column (FIG. 2A). All silica columns were then eluted, and the eluent treated as above. As a comparison, a sample was treated according to the QiaM isolation protocol as described above.

Gel electrophoresis of the resulting nucleic acids demonstrated that, as was demonstrated above, the eluent of just the QiaM protocol contained both mncfDNA and ucsfDNA while the eluent of the first QiaC filtration (QiaC1) contained only mncfDNA (FIG. 2B). The second QiaC filtration (QiaC2) contained a small amount of uscfDNA with minimal mncfDNA, while the final filtration with QiaM (QiaM1) yielded substantial ucsfDNA with no mncfDNA detectable (FIG. 2B).

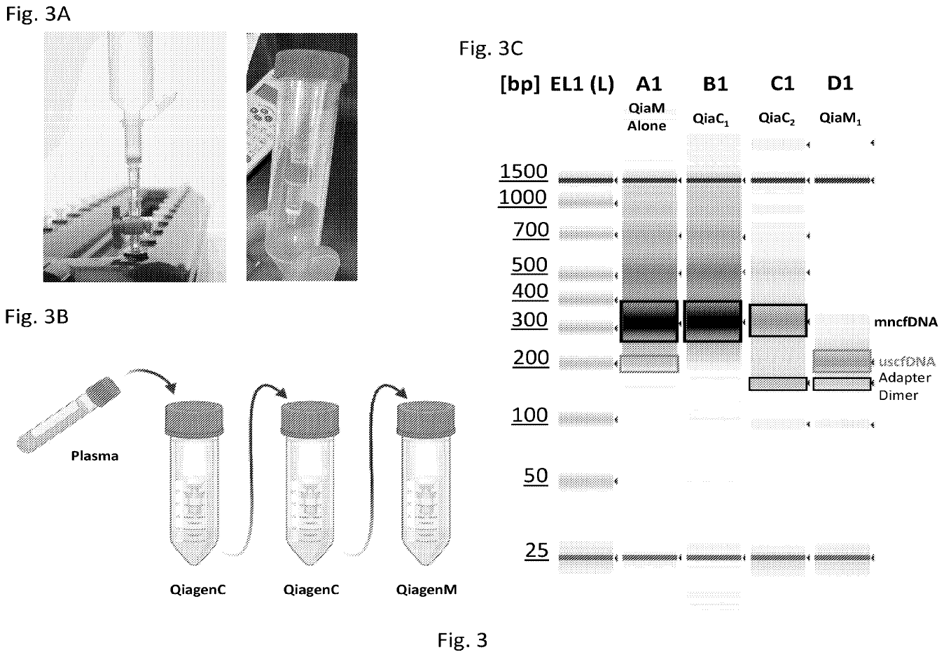

As the high-throughput Qiagen kit uses vacuum filtration, which results in the loss of flowthrough, and the previous methodology required small sample volumes, a larger capacity filtration was developed for rapid processing of larger plasma samples (FIG. 3A). Here, the same three-filtration procedure was followed with 5 mL of plasma and Zymogen Quick-cfDNA Serum and Plasma Kit centrifuge filters with a single QiaM extraction for comparison (FIG. 3B). With this larger filtration, mncfDNA and uscfDNA were again observed in the QiaM filtration and only mncfDNA was observed in the QiaC1 filtration. Here, however, more mncfDNA was observed after the QiaC2 filtration, through significantly less than the initial filtration. After the QiaM1 filtration, once again, only uscfDNA was observed (FIG. 3C).

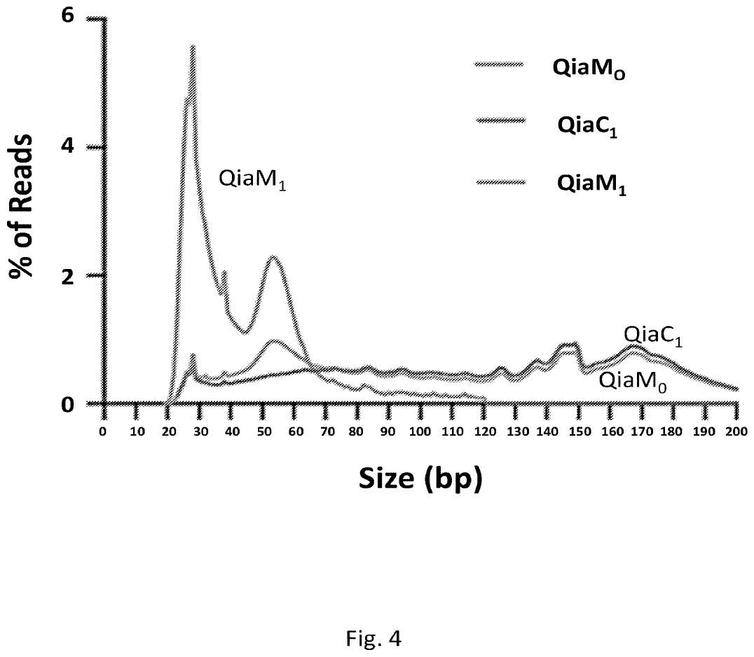

Sequenced libraries were then prepared from the mncfDNA and uscfDNA isolated by this multi-filtration preparation. As seen previously, QiaM-only isolation contains both uscfDNA (25-100 bp) and mncfDNA (120-200 bp). Similarly, QiaC1 elution provided only mncfDNA while the QiaM1 elution exclusively retains uscfDNA (FIG. 4).

### Example 2: Next-generation Seqencing Pipeline to Detect Ultrashort Single-stranded Cell-Free DNA

This invention is based in part on the development of a Next-generation Sequencing (NGS) pipeline to separate and detect ultrashort single-stranded cell-free DNA (uscfDNA). This NGS pipeline is unique in that it is able to separate, detect, and analyze purified samples of ultrashort cell-free ssDNA of 25-75bp separately from the prototypical ~150 bp mononucleosomal cfDNA (mncfDNA). This pipeline combines uscfDNA- and mncfDNA-optimized extraction, ssDNA library construction with unique molecular identifiers, modified clean up-steps to preserve uscfDNA, and an established bioinformatic protocol (FIG. 5). Compared to dsDNA-NGS pipeline it is able to provide significantly greater, and more selective, resolution of uscfDNA.

### Example 3: Ultrashort Single-stranded Cell-free DNA in Biofluids for Disease Detection

This invention encapsulates the detection and analysis of ultra-short single-stranded cell-free DNA (uscfDNA) in patient biofluids as a biomarker for disease. The uscfDNA may potentially contain existing somatic mutations or novel mutations useful for identifying cancer. uscfDNA may contain methylated markers that can be used to identify auto-immunity diseases. The uscfDNA may also be useful as a global biomarker in which its increase concentration may be diagnostic of aberrations in the patient's condition.

### Example 4: Analysis of Ultrashort Single-stranded Cell-free DNA in Patient Saliva for Disease Detection

This invention encapsulates the detection and analysis of ultra-short single-stranded cell-free DNA (uscfDNA) in patient saliva as a biomarker for disease. The uscfDNA may potentially contain existing somatic mutations or novel mutations in the promoter regions useful for identifying cancer. uscfDNA may contain methylated markers that can be used to identify auto-immunity diseases. The uscfDNA may also be useful as a global biomarker in which its increase concentration may be diagnostic of aberrations in the patient's condition.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of separately isolating mono-nucleosomal cfDNA (mncfDNA) and ultrashort single-stranded cell-free DNA (uscfDNA) molecules from a mixed nucleic acid sample, the method comprising the steps of:
a) filtering the sample through a first silica column in the presence of reagents for DNA extraction and purification, wherein the mncfDNA is captured on the column and uscfDNA passes through the column into the flowthrough, and optionally eluting the mncfDNA off the column for downstream analysis;
b) filtering at least a portion of the flowthrough of the filtration of step a) through a second silica column, without additional reagents;
c) adding isopropanol to the flowthrough of the filtration of step b);
d) applying the flowthrough of step c) to a third silica column in the presence of reagents optimized for purification and extraction of microRNA molecules, wherein the uscfDNA is bound to the column;
e) eluting the uscfDNA off the column of step d); and
f) contacting the uscfDNA sample from e) with Solid Phase Reversible Immobilization (SPRI) magnetic beads to clean up the uscfDNA.

2. The method of claim 1, further comprising the step of preparing a sequencing library from the uscfDNA sample from step f).

3. The method of claim 2, further comprising the step of sequencing the library of uscfDNA.

4. The method of claim 1, further comprising contacting the eluted mncfDNA of step a) with SPRI magnetic beads to clean up the mncfDNA.

5. The method of claim 4, further comprising the step of preparing a sequencing library from the extracted mncfDNA.

6. The method of claim 5, further comprising the step of sequencing the library of mncfDNA.

7. The method of claim 1, wherein step b) is performed two or more times.

8. The method of claim 1, wherein the method further comprises a step of lysing a cell or disrupting proteins prior to step a).

9. The method of claim 8, wherein the step of lysing a cell or disrupting proteins comprises:
i) adding proteinase K and SDS to the sample;
ii) incubating the sample for 30 minutes at 60 °C; and
iii) cooling the sample to ambient room temperature.

10. The method of claim 1, wherein the method further comprises a step of isolating genomic DNA from the sample prior to step a).

11. The method of claim 1, wherein the sample is a biological fluid sample.

12. The method of claim 11, wherein the sample is selected from the group consisting of a blood sample, a plasma sample, a saliva sample, a sputum sample, a urine sample, and a liquid biopsy sample.

13. A method of identifying novel biomarkers for diseases or disorders comprising obtaining mncfDNA, uscfDNA, or a combination thereof, from a sample according to the method of claim 1 and analyzing the amount or sequence content of the mncfDNA, uscfDNA, or the combination thereof, to identify novel biomarkers of a disease or disorder.

14. The method of claim 13, wherein the biomarker is selected from the group consisting of a mutation, an indel, a copy number variation, and a methylation marker.

15. The method of claim 13, wherein the biomarker is an increase or decrease in the total amount of mncfDNA, uscfDNA, or a combination thereof in a test sample as compared to a control sample.

16. The method of claim 13, wherein the biomarker is an increase or decrease in the amount of mncfDNA, uscfDNA, or a combination thereof associated with a specific gene in a test sample as compared to a control sample.

17. A method of diagnosing a diseases or disorder in a subject in need thereof, the method comprising obtaining a sample from the subject, isolating mncfDNA, uscfDNA, or a combination thereof from the sample according to the method of claim 1; analyzing the amount or sequence content of the mncfDNA, uscfDNA, or a combination thereof to detect a biomarker of a disease or disorder, and diagnosing the subject as having or at risk of the disease or disorder associated with the identified biomarker.

18. The method of claim 17, wherein the biomarker is selected from the group consisting of a mutation, an indel, a copy number variation, and a methylation marker.

19. The method of claim 17, wherein the biomarker is an increase or decrease in the total amount of mncfDNA, uscfDNA, or a combination thereof in a test sample as compared to a control sample.

20. The method of claim 17, wherein the biomarker is an increase or decrease in the amount of mncfDNA, uscfDNA, or a combination thereof associated with a specific gene in a test sample as compared to a control sample.

21. The method of claim 17, wherein the disease or disorder is selected from the group consisting of an autoimmune disease or disorder, a disease or disorder associated with an infectious agent, and cancer.

22. A kit comprising components for performing the method of claim 1.
